(19) United States
(12) Patent Application Publication
(10) Pub. No.: US 2022/0370723 A1
(43) Pub. Date: Nov. 24, 2022

(54) Dose setting assembly with slack reducing feature

(71) Applicant: Novo Nordisk A/S, Bagsvaerd (DK)

(72) Inventors: Henrik Bengtsson, Taastrup (DK); Bjoern Juul, Broendby (DK); Jan Harald Preuthun, Broenshoej (DK); Zeinab Mahmoudi, Birkeroed (DK); Launits Hoejgaard Olesen, Koebenhavn K (DK)

(21) Appl. No.: 17/772,522

(22) PCT Filed: Dec. 3, 2020
(86) PCT No.: PCT/EP2020/084444

§ 371 Date: Apr. 28, 2022

(30) Foreign Application Priority Data
Dec. 3, 2019 (EP) 19213296.7

(51) Int. Cl. A61M 5/315

(52) U.S. Cl. CPC A61M 5/31553; A61M 2005/3126

(57) ABSTRACT

Add-on device for a drug delivery pen device, comprising a drive member adapted for coupling to a dose dial member of the pen device, a dial member adapted to rotate the drive member in first and second directions to set/adjust a pen dose, a release member axially moveable to actuate a pen device release button, a linear-to-rotational converter mechanism adapted to convert axial movement of the release member to rotational movement of the drive member in a first direction, whereby, when the add-on device is mounted on the drug delivery pen device: the dose dial member of the pen device is biased in the first direction by the drive member thereby taking up rotational slack in the pen device.

The present invention generally relates to medical devices for which efficient and reliable detection of expelled dose amounts is relevant.

### BACKGROUND OF THE INVENTION

In the disclosure of the present invention reference is mostly made to drug delivery devices used e.g. in the treatment of diabetes by subcutaneous delivery of insulin, however, this is only an exemplary use of the present invention.

Drug delivery devices for subcutaneous injections have greatly improved the lives of patients who must self-administer drugs and biological agents. Such drug delivery devices may take many forms, including simple disposable devices that are little more than an ampoule with an injection means or they may be durable devices adapted to be used with prefilled cartridges. Regardless of their form and type, they have proven to be great aids in assisting patients to self-administer injectable drugs and biological agents. They also greatly assist care givers in administering injectable medicines to those incapable of performing self-injections. A common type of drug delivery devices allows a user to set a desired dose size for the drug to be delivered. For a typical mechanical device the dose setting means is in the form of a rotatable dose setting or dial member allowing the user to set (or “dial”) the desired dose size which is then subsequently expelled from the device.

Performing the necessary insulin injection at the right time and in the right size is essential for managing diabetes, i.e. compliance with the specified insulin regimen is important. In order to make it possible for medical personnel to determine the effectiveness of a prescribed dosage pattern, diabetes patients are encouraged to keep a log of the size and time of each injection. However, such logs are normally kept in handwritten notebooks, and the logged information may not be easily uploaded to a computer for data processing. Furthermore, as only events, which are noted by the patient, are logged, the notebook system requires that the patient remembers to log each injection, if the logged information is to have any value in the treatment of the patient's disease. A missing or erroneous record in the log results in a misleading picture of the injection history and thus a misleading basis for the medical personnel's decision making with respect to future medication. Accordingly, it may be desirable to automate the logging of injection information from medication delivery systems.

Though some injection devices integrate this monitoring/acquisition mechanism into the device itself, e.g. as disclosed in US 2009/0318865 and WO 2010/052275, most devices of today are without it. The most widely used devices are purely mechanical devices being either durable or prefilled. The latter devices are to be discarded after being emptied and so inexpensive that it is not cost-effective to build-in electronic data acquisition functionality in the device it-self. Addressing this problem a number of solutions have been proposed which would help a user to generate, collect and distribute data indicative of the use of a given medical device.

For example, WO 2014/037331 describes in a first embodiment an electronic supplementary device (also named “add-on module” or “add-on device”) adapted to be releasably attached to a drug delivery device of the pen type. The device includes a camera and is configured to perform optical character recognition (OCR) on captured images from a rotating scale drum visible through a dosage window on the drug delivery device, thereby to determine a dose of medicament that has been dialled into the drug delivery device. WO 2014/020008 discloses an electronic supplementary device adapted to be releasably attached to a drug delivery device of the pen type. The device includes a camera and is configured to determine scale drum values based on OCR. To properly determine the size of an expelled dose the supplementary device further comprises additional electromechanical sensor means to determine whether a dose size is set, corrected or delivered. A further external device for a pen device is shown in WO 2014/161952.

WO 2019/057911 discloses an add-on dose logging device for a pen-formed drug delivery device, comprising sensor means adapted to capture an amount of rotation of a magnetic member arranged in the drug delivery device, the amount of rotation of the magnetic member corresponding to the amount of drug expelled from a reservoir by the drug delivery device.

Irrespective of the specific nature of the sensor means, dose logging circuitry in general rely on the determination of an amount of rotation for a given component in a drug delivery device, the amount of rotation corresponding to the size of an expelled dose of drug for the given drug delivery device.

Having regard to the above, it is an object of the present invention to provide devices, assemblies and methods allowing efficient, reliable and precise capturing of expelled dose amounts based on the determination of the amount of rotation for a given indicator component or structure. The devices, assemblies and methods may relate to add-on devices adapted to be releasably mounted on a drug delivery device as well as to sensor arrangements formed integrally with a given drug delivery device.

### DISCLOSURE OF THE INVENTION

In the disclosure of the present invention, embodiments and aspects will be described which will address one or more of the above objects or which will address objects apparent from the below disclosure as well as from the description of exemplary embodiments.

The present invention is based on the realization that measuring the amount of out-dosed drug, e.g. units of insulin (IU), can be done in essentially two different ways: directly and (more or less) indirectly.

Units measuring on components directly involved in out-dosing not only have to be able to measure either very small movements or volume changes very accurately and very reliable, but also be able to be either very tolerant or able to compensate for variations in positioning, e.g. of an add-on measuring unit on a given drug delivery device. Components directly involved in out-dosing could be cartridge piston movement or axial movement of the piston rod.

Units measuring on components only indirectly involved, e.g. such as rotation of threaded piston rod, reset-tube and other parts of the dose setting and out-dosing mechanism, have the advantages of usually having to measure larger movements and a known regime of operation. For example, in some pen-formed drug delivery devices the piston rod is known to turn X times 15°, where X is an integer, during out-dosing which means that any measurement between 40° and 50° would indicate a dose size of 3 IU.

However, slack and small variations due to production tolerances typically add up through the mechanism and increase inaccuracy of measurement. Furthermore, these units also face the challenge of add-on units being fitted on each injection device with some, although small, variations in position relative to the components on which they measure. In some cases, the problems are doubled, since the measured start-position may vary significantly as well as the measurements of a given end-position.

A key issue when fitting an add-on measuring device on an existing drug delivery device for injection is that such an add-on system has to measure the change in position of a component from just prior to motion to just after motion has stopped. This means the system will often be designed to measure the movement of a component involved in the dose setting adjustment mechanism. Dose setting adjustment mechanisms are often designed such that the user can dial up a dose size from 0 to a desired dose size, in which case a component is turned a number of “click” increments in a ratchet mechanism.

Each “click” increment corresponds to a given volume of out-dosing, e.g. 1 IU or 0.5 IU, and occurs each time the component is rotated a specific number of degrees. In such ratchet mechanisms, some play or slack between to interacting components are necessary to prevent jamming.

This means that with each interacting pair of components, a little slack must be ensured even in worst-case combinations of production tolerance variations, such that even tight-fit combination production tolerances will result in a little play between the interacting parts. This again means that opposite worst-case combinations will have significantly more play. Often dialing mechanisms involve interactions between 4-6 parts, that form a chain of mechanical parts and the slack between each interacting pair of components adds up to a total slack or play in the dialing mechanism. A further 5-10 interacting components may be involved in the actual out-dosing, adding to the chain of inaccuracy and slack. Since such devices are required to be able to expel a volume with given tolerances, the total slack has to be kept lower than about a third of a unit. Production costs increase significantly with reduced production tolerances and thus devices are mostly designed with maximum allowable slack ensuring performance requirements to be met. Thus, a total slack of up to plus/minus about a quarter of a unit is quite normal for such devices.

However, most drug delivery devices on the market today allows the user to adjust a set dose in a number of increments, e.g. from 0 to 80 IU. Correspondingly, such dose setting mechanisms have to be designed such that an already dialed dose size, i.e. an already dialed number of “clicks” in the ratchet mechanism, can be dialed back to a lower dose setting, in case the user accidently dials too far or re-decides to take a lower dose.

This means that after having dialed up and thereby having absorbed all slack “to one side”, all interacting parts move from engagement in “one side” to the opposite side, before mechanism actually dials back in the ratchet.

This means that the input dial, i.e. the component actually gripped by the user, may have to pick up twice the slack (from plus to minus), which is not an issue during normal us, since the user in most cases will not notice the “silent” slack when the dialing direction is reversed. Furthermore, some mechanisms are designed such that additional slack is introduced by a built-in mechanism to disengage or loosen the ratchet mechanism during normal operation to allow dialing backwards.

The above-described additional slack related to dial-down operation mostly affects the input dial component as the further down through the chain of interacting components, the lesser the components will be affected. The actual dose to be expelled may not be influenced at all. However, in relation to an add-on measuring device, measuring the start- and end-positions of a component close to the actual dial-input component, this may present a problem since the start-position of this component may differ by up to almost a full unit depending on the user having dialed up or down to reach the set dose at dose release. Even with a very high accuracy of measurement, it may be difficult to determine what the actually set dose was and consequently what was out-dosed with an accuracy better than plus/minus one unit, which in some cases may not fulfil the authority requirements for such devices.

Thus, having identified a root course for potential lack of accuracy for a dose logging arrangement relying on determination of rotational movement of a component “close to the dial input member”, an identified problem to be solved is to provide a solution for an add-on device that reduces or eliminates the measuring systems sensitivity to the slack experienced by the component on which measurements are performed between dial-up and dial-down performed by the user. Indeed, such a solution could also be implemented for an integrated dose logging arrangement utilizing the same indicator component.

Thus, in a first general aspect of the invention an assembly is provided in which a build-in mechanism ensures that a torque in the dial-up direction is applied on the dial input component on which the system measures prior to dose release, this ensuring that slack is always picked-up in the same direction during the measurement of start-position, regardless of the last dialed direction by the user.

In a first specific aspect of the invention an add-on device adapted to be releasably mounted on a drug delivery device is provided. The drug delivery device comprises a housing defining a reference axis, a drug reservoir or means for receiving a drug reservoir, and drug expelling means comprising a dose setting member adapted to rotate in a first direction to incrementally set a dose, and rotate in an opposed second direction to incrementally reduce a set dose, the dose setting member being arranged at the proximal end of the housing, and a release member actuatable between a proximal position and a distal position, the proximal position allowing a dose amount to be set, the distal position allowing the drug expelling means to expel a set dose. The add-on device comprises an add-on housing adapted to be releasably attached to the drug delivery device housing in an axially and rotationally non-moveable position, a drive member adapted to be mounted rotationally locked on the dose setting member, an add-on dose setting member coupled to the add-on housing rotatable free but axially locked and adapted to rotate in the first direction to set a dose, and rotate in the opposed second direction to reduce a set dose, and an actuatable add-on release member. The actuatable add-on release member is axially moveable relative to the add-on dose setting member between (i) a proximal dose setting position in which the add-on dose setting member can be operated to rotate the drive member and thereby, when mounted on the dose setting member, set a dose, and (ii) a distal dose expelling position in which the release member, when the add-on device is mounted on the drug delivery device, is moved to its distal position to release a set dose. The add-on device further comprises a linear-to-rotational converter mechanism adapted to convert axial movement of the add-on release member when the add-on release member is moved from the proximal position towards the distal position to rotational movement of the drive member in the first direction, whereby, when the add-on device is mounted on the drug delivery device, the dose setting member is biased in the first direction by the drive member when the add-on release member is moved from the proximal position towards the distal position.

By this arrangement it can be ensured that slack in the drug delivery device dose setting mechanism can be picked-up in the same direction during the measurement of a start-position, regardless of last dialed direction by the user.

In order to prevent that rotational movement of the drive member results in the dose setting member being moved an increment and thus the set dose being changed, the maximum rotational movement of the drive member should be designed to be less than the rotational movement required to change the set dose by an increment in the drug delivery device on which the add-on device is adapted to be mounted.

However, to achieve the above object the add-on device would have to be manufactured with close tolerances just at it would have to be assured that the maximum slack in the dose setting mechanism (by a margin) would be less than the amount of rotation required to shift the dose setting member an increment.

Addressing this issue, the add-on device in an exemplary embodiment comprises a torque limiter preventing that a torque above a pre-set level can be transferred to the drive member when the add-on release member is moved from the proximal position towards the distal position.

In this way an add-on device can be provided which with high certainty would provide an amount of rotation of the drive member corresponding to the maximally expected slack in the drug expelling means, and at the same time reduces the risk of changing the set dose.

As is well known to the person skilled in the art, a torque limiter is an automatic device/mechanism that protects mechanical equipment from damage by mechanical overload or, as disclosed above, undesired movement. From textbooks on mechanical design numerous types of torque limiters are known. For example, in exemplary embodiments the torque limiter may comprise a flexible element adapted to deform when a torque above the pre-set level has been transferred to the drive member. Indeed, a (very) small amount of deformation will take place before the pre-set level has been reached. Alternatively, the torque limiter may be in the form of a ratchet torque limiter.

In exemplary embodiments the rotational movement is generated by a cam-follower mechanism. It is to be noted that in the context of the present invention the term “cam-follower mechanism” covers both the “traditional” embodiment in which a cam (or slot or track) member is actively moved with the follower being the passively moved member, as well as the “reversed” embodiment in which the “follower” is actively moved with the cam member being the passively moved member. Alternatively, the linear-to-rotational converter mechanism may comprise “free” inclined surfaces moving relative to each other.

In specific embodiments the cam-follower mechanism comprises a track and a follower arranged in the track, the track having a radial component, the follower and track moving axially relative to each other when the add-on release member is moved from the proximal position towards the distal position.

In a specific embodiment, the follower comprises a flexible arm adapted to bend when a torque above a pre-set level has been transferred to the drive member, this providing a cam-follower mechanism with an integrated torque limiter.

Alternatively, the torque-limiter preventing that a rotational force above a pre-determined level is applied to the dose setting member via the drive member is a ratchet torque limiter, i.e. a clutch that will slip or “jump” when the desired amount of torque has been applied.

Irrespective of the specific design of the linear-to-rotational converter mechanism and the torque limiter, the drug expelling means may comprise an indicator adapted to rotate during expelling of a dose amount, the amount of rotations being indicative of the size of the expelled dose amount. The add-on device is correspondingly provided with sensor means operatable to detect the amount of rotation of the indicator during expelling of a dose amount. When the linear-to-rotational converter mechanism has been actuated prior to the sensor means being operated it can be assured that rotational slack in the drug expelling means has been removed by rotation of the dose setting member in the first dial-up direction.

In exemplary embodiments the add-on device comprises an actuator structure adapted to move axially and engage the release member when the add-on release member is actuated, wherein electronic sensor circuitry is coupled to and moves axially with the actuator structure. The actuator structure may be in the form of an electronic assembly, e.g. unit or module, comprising the electronic sensor circuitry, e.g. in the form of sensor components, a processor, memory, wireless communication means and a battery.

Alternatively, the drug delivery device may be adapted for mounting of a dose logging add-on device comprising sensor means operatable to detect the amount of rotation of an indicator during expelling of a dose amount, e.g. in the form of numerals on the scale drum.

In a specific embodiment the indicator comprises a plurality of dipole magnets, and the sensor means comprises a plurality of magnetometers arranged non-rotational relative to the housing in a mounted state and adapted to determine magnetic field values from the plurality of dipole magnets, and processor means configured to determine on the basis of measured values from the plurality of magnetometers a rotational position and/or a rotational movement of the indicator. Such sensor means may be incorporated in the anti-slack add-on device or in a separate additional sensor add-on device.

In a further aspect of the invention an assembly is provided comprising a drug delivery device and a corresponding add-on device as disclosed above.

In a yet further aspect of the invention, a unitary drug delivery device with integrated slack counter-acting means as disclosed above is provided, such a device comprising a housing defining a reference axis, a drug reservoir or means for receiving a drug reservoir, and drug expelling means. The drug expelling means comprising an inner dose setting member adapted to rotate in a first direction to set a dose, and rotate in an opposed second direction to reduce a set dose, an inner release member actuatable between a proximal position and a distal position, the proximal position allowing a dose amount to be set, the distal position allowing the drug expelling means to expel a set dose, and an indicator adapted to rotate during expelling of a dose amount, the amount of rotation being indicative of the size of the expelled dose amount. The device further comprises a user dose setting member coupled to the housing rotatable free but axially locked and adapted to rotate in the first direction to set a dose, and rotate in the opposed second direction to reduce a set dose, an actuatable user release member axially moveable relative to the user dose setting member between (i) a proximal dose setting position in which the user dose setting member can be operated to rotate the inner dose setting member to set a dose, and (ii) a distal dose expelling position in which the inner release member is moved to its distal position to release a set dose, as well as a linear-to-rotational converter mechanism adapted to convert axial movement of the user release member, when the user release member is moved from the proximal position towards the distal position, to rotational movement of the inner dose setting member in the first direction. Hereby the inner dose setting member is biased in the first direction when the drug expelling means is released. The biasing force applied by to the inner dose setting member may be controlled by a torque limiter to a maximum level assuring that a set dose will not be changed.

The drug delivery device may be provided with integrated sensor means operatable to detect the amount of rotation of the indicator during expelling of a dose amount. Alternatively, the drug delivery device may be adapted for mounting of a dose logging add-on device comprising sensor means operatable to detect the amount of rotation of the indicator during expelling of a dose amount.

In specific embodiments the indicator comprises a plurality of dipole magnets, and the sensor means comprises a plurality of magnetometers arranged non-rotational relative to the housing in a mounted state and adapted to determine magnetic field values from the plurality of dipole magnets, and processor means configured to determine on the basis of measured values from the plurality of magnetometers a rotational position and/or a rotational movement of the indicator.

Irrespective of the specific design of the biasing arrangement, the drug expelling means may comprise an indicator adapted to rotate during expelling of a dose amount, the amount of rotations being indicative of the size of the expelled dose amount. The add-on device is correspondingly provided with sensor means operatable to detect the amount of rotation of the indicator during expelling of a dose amount. When the linear-to-rotational converter mechanism has been actuated prior to the sensor means being operated it can be assured that rotational slack in the drug expelling means has been removed by rotation of the dose setting member in the first dial-up direction.

As used herein, the term “insulin” is meant to encompass any drug-containing flowable medicine capable of being passed through a delivery means such as a cannula or hollow needle in a controlled manner, such as a liquid, solution, gel or fine suspension, and which has a blood glucose controlling effect, e.g. human insulin and analogues thereof as well as non-insulins such as GLP-1 and analogues thereof. In the description of exemplary embodiments reference will be made to the use of insulin, however, the described module could also be used to create logs for other types of drug, e.g. growth hormone.

### BRIEF DESCRIPTION OF THE DRAWINGS

In the following embodiments of the invention will be described with reference to the drawings, wherein

FIG. 1A shows a pen device,

FIG. 1B shows the pen device of FIG. 1A with the pen cap removed,

FIG. 2 shows in an exploded view the components of the pen device of FIG. 1A,

FIGS. 3A and 3B show in sectional views an expelling mechanism in two states,

FIGS. 4A and 4B show a schematic representation of an add-on device and a drug delivery device,

FIGS. 5A and 5B respectively show an exemplary drug delivery device and a corresponding add-on device adapted to be mounted thereon as shown in FIG. 4B,

FIG. 6A shows in a partial transparent view a first embodiment of an add-on dose logging device comprising a slack counter-acting mechanism,

FIG. 6B shows the add-on device of FIG. 6A in a cross-sectional view,

FIGS. 7A-7D show individual components forming part of the add-on device of FIG. 6A,

FIGS. 8A-8H show in a partial cut-away representation the add-on device of FIG. 6A in different operational states when mounted on the drug delivery device of FIG. 5A,

FIG. 9A shows a second embodiment of an add-on dose logging device comprising a slack counter-acting mechanism,

FIG. 9B shows an inner assembly of the add-on device of FIG. 9A

FIG. 9C shows the add-on device of FIG. 9A in a cross-sectional view,

FIGS. 10A-10D show individual components forming part of the add-on device of FIG. 9A,

FIGS. 11A-11E show in a partial cut-away representation the add-on device of FIG. 9A in different operational states when mounted on the drug delivery device of FIG. 5A, and

FIG. 12 shows the assembly of FIG. 11A in an alternative representation and view.

In the figures like structures are mainly identified by like reference numerals.

### DESCRIPTION OF EXEMPLARY EMBODIMENTS

When in the following terms such as “upper” and “lower”, “right” and “left”, “horizontal” and “vertical” or similar relative expressions are used, these only refer to the appended figures and not necessarily to an actual situation of use. The shown figures are schematic representations for which reason the configuration of the different structures as well as their relative dimensions are intended to serve illustrative purposes only. When the term member or element is used for a given component it generally indicates that in the described embodiment the component is a unitary component, however, the same member or element may alternatively comprise a number of sub-components just as two or more of the described components could be provided as unitary components, e.g. manufactured as a single injection moulded part. The term “assembly” does not imply that the described components necessarily can be assembled to provide a unitary or functional assembly during a given assembly procedure but is merely used to describe components grouped together as being functionally more closely related.

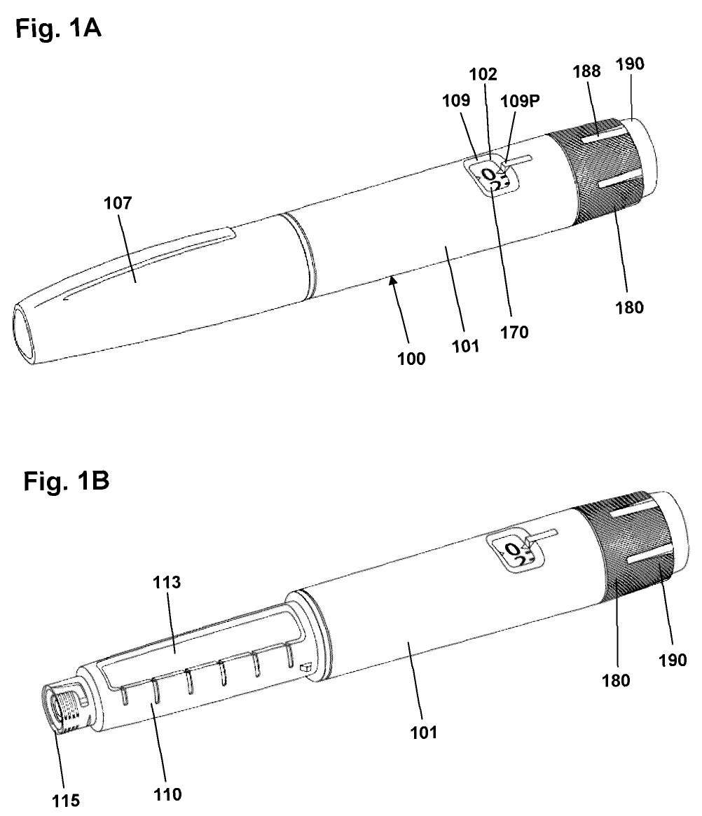

Before turning to embodiments of the present invention per se, an example of a prefilled drug delivery will be described, such a device providing the basis for the exemplary embodiments of the present invention. Although the pen-formed drug delivery device 100 shown in FIGS. 1-3 may represent a “generic” drug delivery device, the actually shown device is a FlexTouch® prefilled drug delivery pen as manufactured and sold by Novo Nordisk A/S, Bagsværd, Denmark.

The pen device 100 comprises a cap part 107 and a main part having a proximal body or drive assembly portion with a housing 101 in which a drug expelling mechanism is arranged or integrated, and a distal cartridge holder portion in which a drug-filled transparent cartridge 113 with a distal needle-penetrable septum is arranged and retained in place by a non-removable cartridge holder attached to the proximal portion, the cartridge holder having openings allowing a portion of the cartridge to be inspected as well as distal coupling means 115 allowing a needle assembly to be releasably mounted. The cartridge is provided with a piston driven by a piston rod forming part of the expelling mechanism and may for example contain an insulin, GLP-1 or growth hormone formulation. A proximal-most rotatable dose setting member 180 with a number of axially oriented grooves 188 serves to manually set a desired dose of drug shown in display window 102 and which can then be expelled when the button 190 is actuated. As will be apparent from the below description, the shown axially oriented grooves 188 may be termed “drive grooves”. The dose setting member 180 has a generally cylindrical outer surface 181 (i.e. the dose setting member may be slightly tapered) which in the shown embodiment is textured by comprising a plurality of axially oriented fine grooves to improve finger grip during dose setting. The window is in the form of an opening in the housing surrounded by a chamfered edge portion 109 and a dose pointer 109P, the window allowing a portion of a helically rotatable indicator member 170 (scale drum) to be observed. Depending on the type of expelling mechanism embodied in the drug delivery device, the expelling mechanism may comprise a spring as in the shown embodiment which is strained during dose setting and then released to drive the piston rod when the release button is actuated. Alternatively the expelling mechanism may be fully manual in which case the dose member and the actuation button move proximally during dose setting corresponding to the set dose size, and then is moved distally by the user to expel the set dose, e.g. as in a FlexPen® manufactured and sold by Novo Nordisk A/S.

Although FIG. 1 shows a drug delivery device of the prefilled type, i.e. it is supplied with a pre-mounted cartridge and is to be discarded when the cartridge has been emptied, in alternative embodiments the drug delivery device may be designed to allow a loaded cartridge to be replaced, e.g. in the form of a “rear-loaded” drug delivery device in which the cartridge holder is adapted to be removed from the device main portion, or alternatively in the form of a “frontloaded” device in which a cartridge is inserted through a distal opening in the cartridge holder which is non-removable attached to the main part of the device.

As the invention relates to electronic circuitry adapted to interact with a drug delivery device, an exemplary embodiment of such a device will be described for better understanding of the invention.

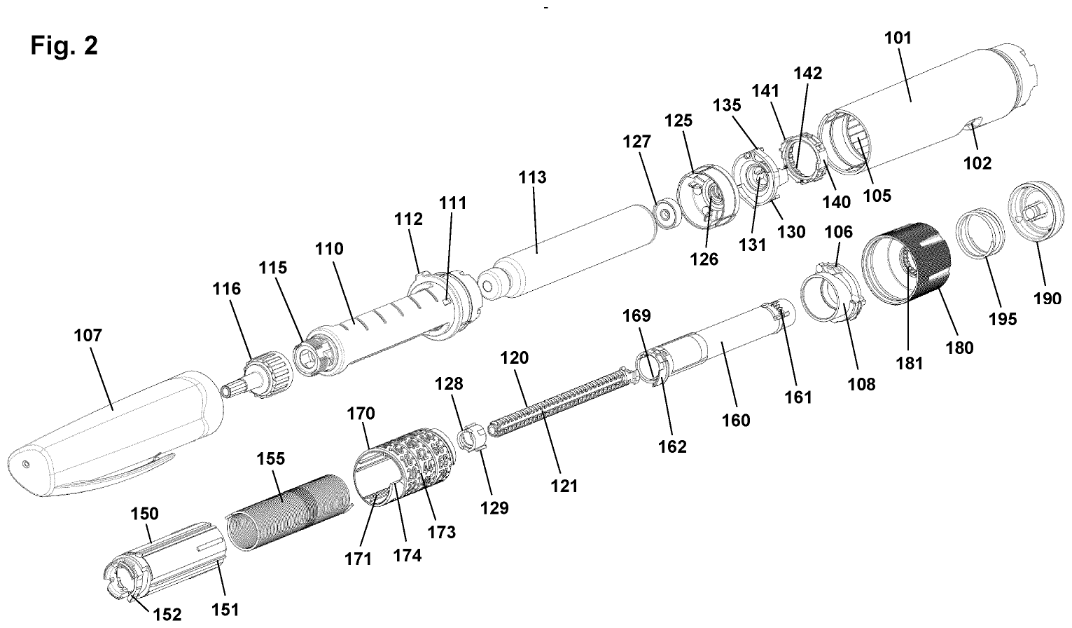

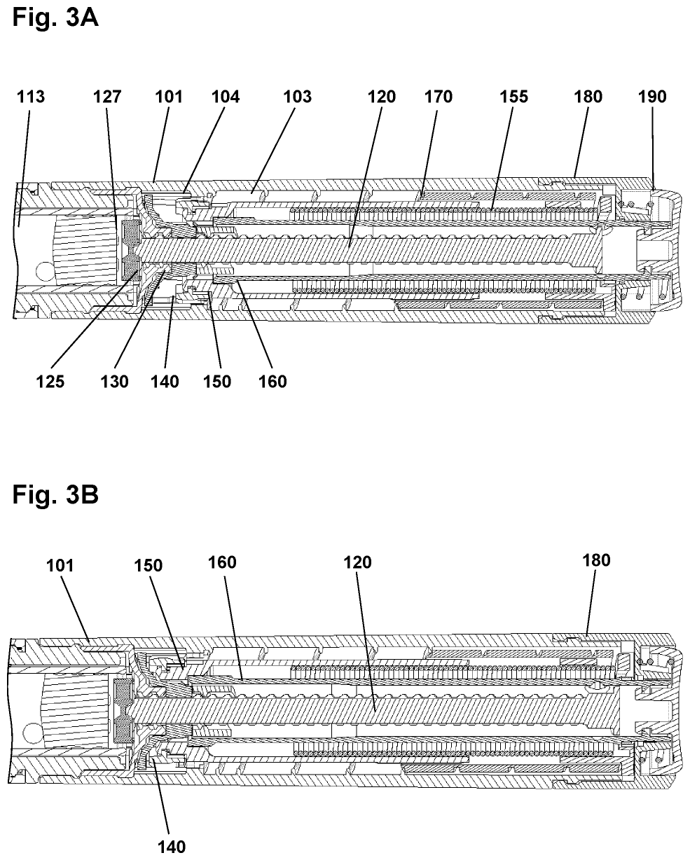

FIG. 2 shows an exploded view of the pen-formed drug delivery device 100 shown in FIG. 1. More specifically, the pen comprises a tubular housing 101 with a window opening 102 and onto which a cartridge holder 110 is fixedly mounted, a drug-filled cartridge 113 being arranged in the cartridge holder. The cartridge holder is provided with distal coupling means 115 allowing a needle assembly 116 to be releasable mounted, proximal coupling means in the form of two opposed protrusions 111 allowing a cap 107 to be releasable mounted covering the cartridge holder and a mounted needle assembly, as well as a protrusion 112 preventing the pen from rolling on e.g. a table top. In the housing distal end a nut element 125 is fixedly mounted, the nut element comprising a central threaded bore 126, and in the housing proximal end a spring base member 108 with a central opening is fixedly mounted. A drive system comprises a threaded piston rod 120 having two opposed longitudinal grooves and being received in the nut element threaded bore, a ring-formed piston rod drive element 130 rotationally arranged in the housing, and a ring-formed clutch element 140 which is in rotational engagement with the drive element (see below), the engagement allowing axial movement of the clutch element. The clutch element is provided with outer spline elements 141 adapted to engage corresponding splines 104 (see FIG. 3B) on the housing inner surface, this allowing the clutch element to be moved between a rotationally locked proximal position, in which the splines are in engagement, and a rotationally free distal position in which the splines are out of engagement. As just mentioned, in both positions the clutch element is rotationally locked to the drive element. The drive element comprises a central bore with two opposed protrusions 131 in engagement with the grooves on the piston rod whereby rotation of the drive element results in rotation and thereby distal axial movement of the piston rod due to the threaded engagement between the piston rod and the nut element. The drive element further comprises a pair of opposed circumferentially extending flexible ratchet arms 135 adapted to engage corresponding ratchet teeth 105 arranged on the housing inner surface. The drive element and the clutch element comprise cooperating coupling structures rotationally locking them together but allowing the clutch element to be moved axially, this allowing the clutch element to be moved axially to its distal position in which it is allowed to rotate, thereby transmitting rotational movement from the dial system (see below) to the drive system. The interaction between the clutch element, the drive element and the housing will be shown and described in greater detail with reference to FIGS. 3A and 3B.

On the piston rod an end-of-content (EOC) member 128 is threadedly mounted and on the distal end a washer 127 is rotationally mounted. The EOC member comprises a pair of opposed radial projections 129 for engagement with the reset tube (see below).

The dial system comprises a ratchet tube 150, a reset tube 160, a scale drum 170 with an outer helically arranged pattern forming a row of dose indicia, a user-operated dial member 180 for setting a dose of drug to be expelled, a release button 190 and a torque spring 155 (see FIG. 3). The dial member is provided with a circumferential inner teeth structure 181 engaging a number of corresponding outer teeth 161 arranged on the reset tube, this providing a dial coupling which is in an engaged state when the reset tube is in a proximal position during dose setting and in a disengaged state when the reset tube is moved distally during expelling of a dose. The reset tube is mounted axially locked inside the ratchet tube but is allowed to rotate a few degrees (see below). The reset tube comprises on its inner surface two opposed longitudinal grooves 169 adapted to engage the radial projections 129 of the EOC member, whereby the EOC can be rotated by the reset tube but is allowed to move axially. The clutch element is mounted axially locked on the outer distal end portion of the ratchet tube 150, this providing that the ratchet tube can be moved axially in and out of rotational engagement with the housing via the clutch element. The dial member 180 is mounted axially locked but rotationally free on the housing proximal end, the dial ring being under normal operation rotationally locked to the reset tube (see below), whereby rotation of the dial ring results in a corresponding rotation of the reset tube 160 and thereby the ratchet tube. The release button 190 is axially locked to the reset tube but is free to rotate. A return spring 195 provides a proximally directed force on the button and the thereto mounted reset tube. The scale drum 170 is arranged in the circumferential space between the ratchet tube and the housing, the drum being rotationally locked to the ratchet tube via cooperating longitudinal splines 151, 171 and being in rotational threaded engagement with the inner surface of the housing via cooperating thread structures 103, 173, whereby the row of numerals passes the window opening 102 in the housing when the drum is rotated relative to the housing by the ratchet tube. The torque spring is arranged in the circumferential space between the ratchet tube and the reset tube and is at its proximal end secured to the spring base member 108 and at its distal end to the ratchet tube, whereby the spring is strained when the ratchet tube is rotated relative to the housing by rotation of the dial member. A ratchet mechanism with a flexible ratchet arm 152 is provided between the ratchet tube and the clutch element, the latter being provided with an inner circumferential teeth structures 142, each tooth providing a ratchet stop such that the ratchet tube is held in the position to which it is rotated by a user via the reset tube when a dose is set. In order to allow a set dose to be reduced a ratchet release mechanism 162 is provided on the reset tube and acting on the ratchet tube, this allowing a set dose to be reduced by one or more ratchet increments by turning the dial member in the opposite direction, the release mechanism being actuated when the reset tube is rotated the above-described few degrees relative to the ratchet tube.

Having described the different components of the expelling mechanism and their functional relationship, operation of the mechanism will be described next with reference mainly to FIGS. 3A and 3B.

The pen mechanism can be considered as two interacting systems, a dose system and a dial system, this as described above. During dose setting the dial mechanism rotates and the torsion spring is loaded. The dose mechanism is locked to the housing and cannot move. When the push button is pushed down, the dose mechanism is released from the housing and due to the engagement to the dial system the torsion spring will now rotate back the dial system to the starting point and rotate the dose system along with it.

The central part of the dose mechanism is the piston rod 120, the actual displacement of the plunger being performed by the piston rod. During dose delivery, the piston rod is rotated by the drive element 130 and due to the threaded interaction with the nut element 125 which is fixed to the housing, the piston rod moves forward in the distal direction. Between the rubber piston and the piston rod, the piston washer 127 is placed which serves as an axial bearing for the rotating piston rod and evens out the pressure on the rubber piston. As the piston rod has a non-circular cross section where the piston rod drive element engages with the piston rod, the drive element is locked rotationally to the piston rod, but free to move along the piston rod axis. Consequently, rotation of the drive element results in a linear forwards movement of the piston. The drive element is provided with small ratchet arms 134 which prevent the drive element from rotating clockwise (seen from the push button end). Due to the engagement with the drive element, the piston rod can thus only move forwards. During dose delivery, the drive element rotates anti-clockwise and the ratchet arms 135 provide the user with small clicks due to the engagement with the ratchet teeth 105, e.g. one click per unit of insulin expelled.

Turning to the dial system, the dose is set and reset by turning the dial member 180. When turning the dial, the reset tube 160, the EOC member 128, the ratchet tube 150 and the scale drum 170 all turn with it due to the dial coupling being in the engaged state. As the ratchet tube is connected to the distal end of the torque spring 155, the spring is loaded. During dose setting, the arm 152 of the ratchet performs a dial click for each unit dialled due to the interaction with the inner teeth structure 142 of the clutch element. In the shown embodiment the clutch element is provided with 24 ratchet stops providing 24 clicks (increments) for a full 360 degrees rotation relative to the housing. The spring is preloaded during assembly which enables the mechanism to deliver both small and large doses within an acceptable speed interval. As the scale drum is rotationally engaged with the ratchet tube, but movable in the axial direction and the scale drum is in threaded engagement with the housing, the scale drum will move in a helical pattern when the dial system is turned, the number corresponding to the set dose being shown in the housing window 102.

The ratchet 152, 142 between the ratchet tube and the clutch element 140 prevents the spring from turning back the parts. During resetting, the reset tube moves the ratchet arm 152, thereby releasing the ratchet click by click, one click corresponding to 1 IU in the described embodiment. More specifically, when the dial member is turned clockwise, the reset tube simply rotates the ratchet tube allowing the arm of the ratchet to freely interact with the teeth structures 142 in the clutch element. When the dial member is turned counter-clockwise, the reset tube interacts directly with the ratchet click arm forcing the click arm towards the centre of the pen away from the teeth in the clutch, thus allowing the click arm on the ratchet to move “one click” backwards due to torque caused by the loaded spring.

To deliver a set dose, the push button 190 is pushed in the distal direction by the user as shown in FIG. 3B. The dial coupling 161, 181 disengages and the reset tube 160 decouples from the dial member and subsequently the clutch element 140 disengages the housing splines 104. Now the dial mechanism returns to “zero” together with the drive element 130, this leading to a dose of drug being expelled. It is possible to stop and start a dose at any time by releasing or pushing the push button at any time during drug delivery. A dose of less than 5 IU normally cannot be paused, since the rubber piston is compressed very quickly leading to a compression of the rubber piston and subsequently delivery of insulin when the piston returns to the original dimensions.

The EOC feature prevents the user from setting a larger dose than left in the cartridge. The EOC member 128 is rotationally locked to the reset tube, which makes the EOC member rotate during dose setting, resetting and dose delivery, during which it can be moved axially back and forth following the thread of the piston rod. When it reaches the proximal end of the piston rod a stop is provided, this preventing all the connected parts, including the dial member, from being rotated further in the dose setting direction, i.e. the now set dose corresponds to the remaining drug content in the cartridge.

The scale drum 170 is provided with a distal stop surface 174 adapted to engage a corresponding stop surface on the housing inner surface, this providing a maximum dose stop for the scale drum preventing all the connected parts, including the dial member, from being rotated further in the dose setting direction. In the shown embodiment the maximum dose is set to 80 IU. Correspondingly, the scale drum is provided with a proximal stop surface adapted to engage a corresponding stop surface on the spring base member, this preventing all the connected parts, including the dial member, from being rotated further in the dose expelling direction, thereby providing a “zero” stop for the entire expelling mechanism.

To prevent accidental over-dosage in case something should fail in the dialling mechanism allowing the scale drum to move beyond its zero-position, the EOC member serves to provide a security system. More specifically, in an initial state with a full cartridge the EOC member is positioned in a distal-most axial position in contact with the drive element. After a given dose has been expelled the EOC member will again be positioned in contact with the drive element. Correspondingly, the EOC member will lock against the drive element in case the mechanism tries to deliver a dose beyond the zero-position. Due to tolerances and flexibility of the different parts of the mechanism the EOC will travel a short distance allowing a small “overdose” of drug to be expelled, e.g. 3-5 IU of insulin.

The expelling mechanism further comprises an end-of-dose (EOD) click feature providing a distinct feedback at the end of an expelled dose informing the user that the full amount of drug has been expelled. More specifically, the EOD function is made by the interaction between the spring base and the scale drum. When the scale drum returns to zero, a small click-arm 106 on the spring base is forced backwards by the progressing scale drum. Just before “zero” the arm is released and the arm hits a countersunk surface on the scale drum.

The shown mechanism is further provided with a torque limiter in order to protect the mechanism from overload applied by the user via the dial member. This feature is provided by the interface between the dial member and the reset tube which as described above are rotationally locked to each other. More specifically, the dial member is provided with circumferential inner teeth structure 181 engaging a number of corresponding outer teeth 161, the latter being arranged on a flexible carrier portion of the reset tube. The reset tube teeth are designed to transmit a torque of a given specified maximum size, e.g. 150-300 Nmm, above which the flexible carrier portion and the teeth will bend inwards and make the dial member turn without rotating the rest of the dial mechanism. Thus, the mechanism inside the pen cannot be stressed at a higher load than the torque limiter transmits through the teeth.

Having described the working principles of a mechanical drug delivery device, embodiments of the present invention will be described.

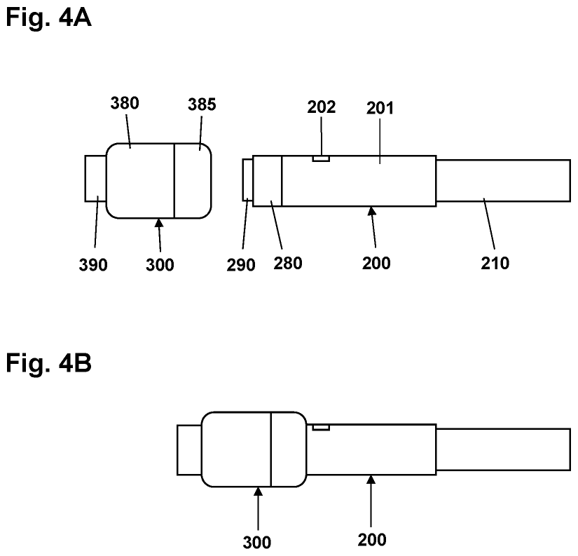

FIGS. 4A and 4B show a schematic representation of a first assembly of a pre-filled pen-formed drug delivery device 200 and a therefor adapted add-on dose logging device 300. The add-on device is adapted to be mounted on the proximal end portion of the pen device housing and is provided with dose setting and dose release means 380 covering the corresponding means on the pen device in a mounted state as shown in FIG. 4B. In the shown embodiment the add-on device comprises a coupling portion 385 adapted to be mounted axially and rotationally locked on the drug delivery housing. The add-on device comprises a rotatable dose setting member 380 which during dose setting is directly or indirectly coupled to the pen dose setting member 280 such that rotational movement of the add-on dose setting member in either direction is transferred to the pen dose setting member. In order to reduce influences from the outside during dose expelling and dose size determination, the outer add-on dose setting member 380 may be rotationally decoupled from the pen dose setting member 280 during dose release and expelling. The add-on device further comprises a dose release member 390 which can be moved distally to thereby actuate the pen release member 290.

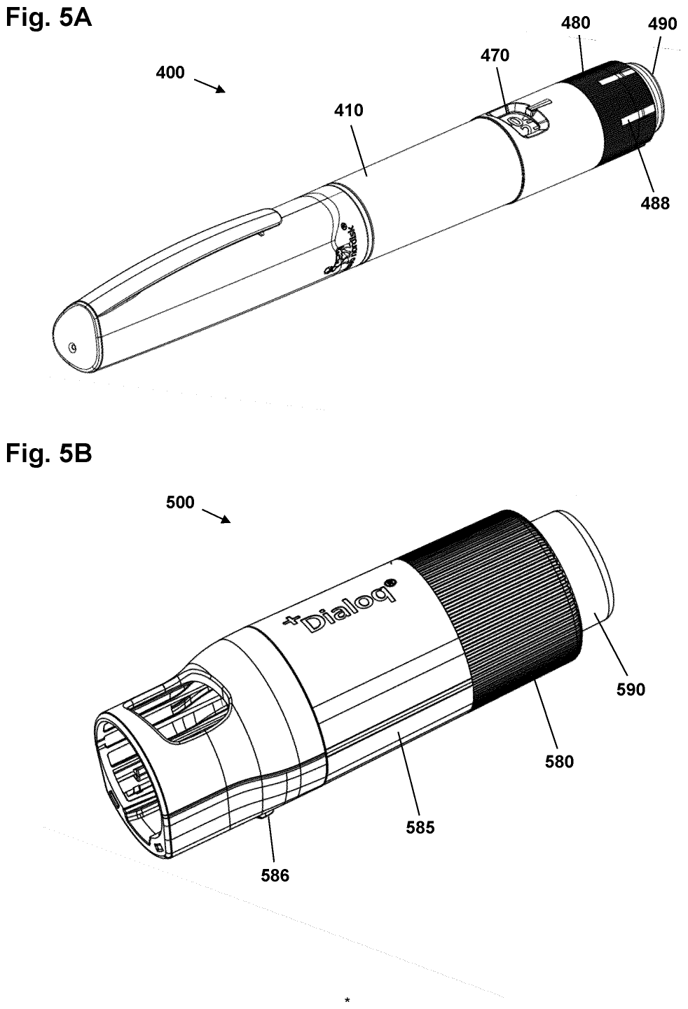

Turning to FIGS. 5A and 5B a first exemplary specific embodiment of an add-on dose logging device 500 adapted to be mounted on a pen-formed drug delivery device 400 is shown, the pen device essentially corresponding to a FlexTouch® prefilled drug delivery pen with the shown add-on dose logging device being branded as “Dialoq®”. The add-on device 500 essentially corresponds to the above-described add-on dose logging device 300 and thus comprises a housing portion 585 adapted to be mounted axially and rotationally locked on the drug delivery housing via releasable coupling means 586, the housing being provided with a window 587 allowing the pen scale drum 470 to be observed during dose setting. The add-on device comprises a rotatable dose setting member 580 which during dose setting is coupled to the pen dose setting member 480 such that rotational movement of the add-on dose setting member in either direction is transferred to the pen dose setting member. The add-on device further comprises a dose release member 590 which can be moved distally to thereby actuate the pen release member 490.

In order to determine the size of an expelled dose amount of drug, the pen device may be provided with magnetic identifiers adapted to rotate during dose expelling and the add-on device may correspondingly be provided with sensor circuitry allowing the amount of rotation to be captured and thereby the expelled dose size to be determined. A number of embodiments based on this concept is disclosed and described in detail in WO 2019/162235 which is hereby incorporated by reference.

In summary, the add-on device disclosed in WO 2019/162235 comprises an outer assembly being releasably attachable to the drug delivery device housing, and an inner assembly. The outer assembly comprises an add-on dose setting member 580, and an add-on release member 590 axially moveable relative to the add-on dose setting member between a dose setting state and a dose expelling state. The inner assembly comprises an inner dose setting member adapted to engage the dose setting member 480, sensor means adapted to detect the amount of rotation of the indicator during expelling of a dose amount, and an actuator coupled to the add-on release member and being axially moveable between a proximal position and a distal position relative to the inner dose setting member, the actuator being adapted to engage and actuate the pen device release member 490 when moved distally. The sensor circuitry, e.g. in the form of an electronic module, may form part of the actuator (and thus move axially therewith) and be coupled non-rotationally to the inner dose setting member to prevent rotation during dose expelling. The sensor circuitry will typically be activated from a sleep state when the user actuates and axially moves the add-on release member 590.

As discussed above, having identified a root course for potential lack of accuracy for a dose logging arrangement relying on determination of rotational movement of a component “close to the dial input member”, an identified problem to be solved is to provide a solution for an add-on device that reduces or eliminates the measuring systems sensitivity to the slack experienced by the component on which measurements are performed (e.g. an indicator member provided with magnets) between dial-up and dial-down performed by the user.

Correspondingly, in the following exemplary embodiments assemblies are provided in which a build-in mechanism ensures that a torque in the dial-up direction is applied on the dial-input component on which the system measures prior to dose release, this ensuring that slack is always picked-up in the same direction during the measurement of start-position, regardless of last dialed direction by the user.

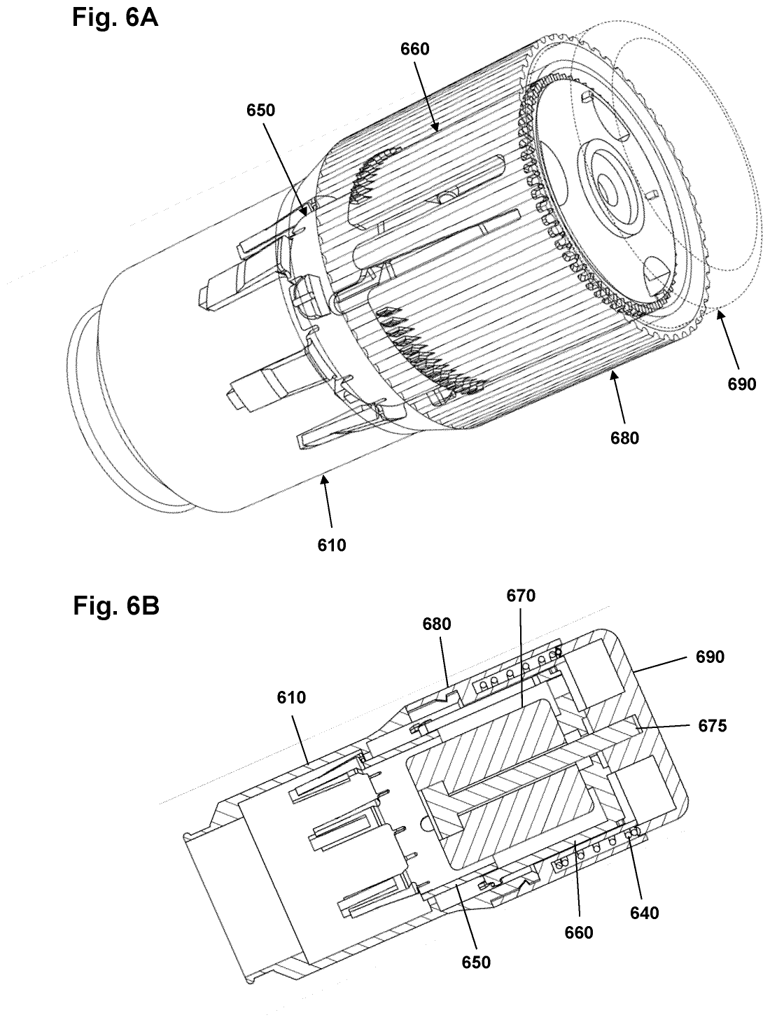

Turning to FIGS. 6A and 6B a first exemplary embodiment of a slack counter-acting mechanism suitable for incorporation into an add-on dose logging device of the general type disclosed in FIG. 5B will be described.

FIG. 6A shows in a partial “transparent” view an add-on dose logging device 600 comprising an outer assembly (shown in “transparent”) and a thereto coupled inner assembly. The outer assembly comprises a housing member 610 adapted to be coupled to the pen housing, a thereto rotationally coupled add-on dial member 680 as well as an add-on dose release member (button) 690. The inner assembly comprises a drive member 650 adapted to non-rotationally engage the pen device dial member, a rotor member 660 arranged to transfer rotational movement from the dial member 680 to the drive member 650 during dose setting and to provide slack counter-acting rotation during dose release, as well as an electronic sensor assembly (ESA) 670 (see FIG. 6B) arranged in the hollow interior of the rotor member 660. In FIG. 6B the ESA 670 is shown arranged inside the rotor member, the latter having an opening in the proximal end allowing a securing pin 675 to connect the add-on release member 690 with the ESA 670, such that all parts are able to rotate relative to each other and move slightly in axial direction.

Before describing the working principle of the add-on device 600 the individual cooperating members will be described in greater detail.

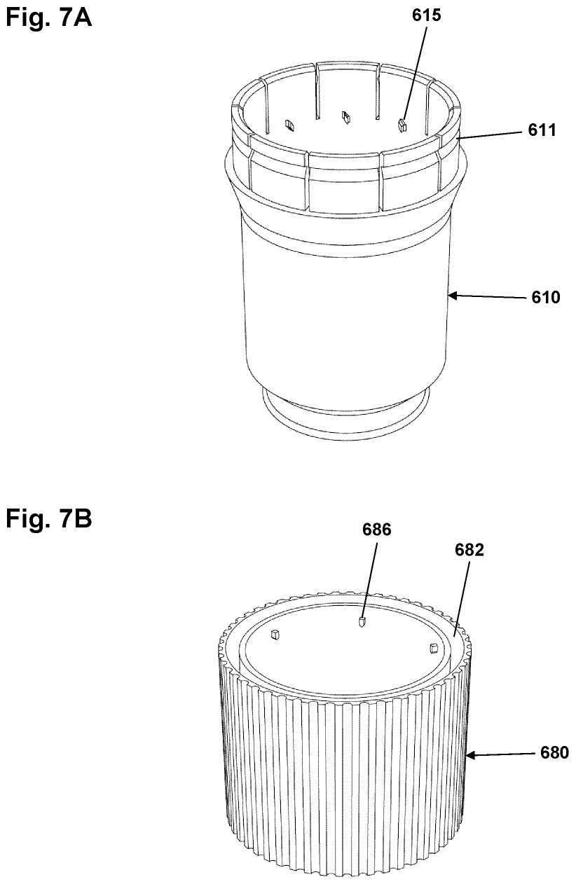

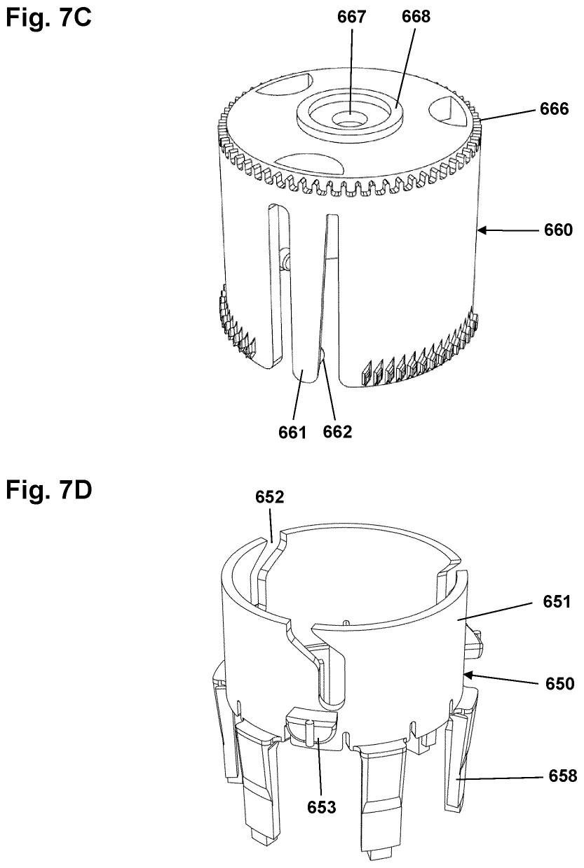

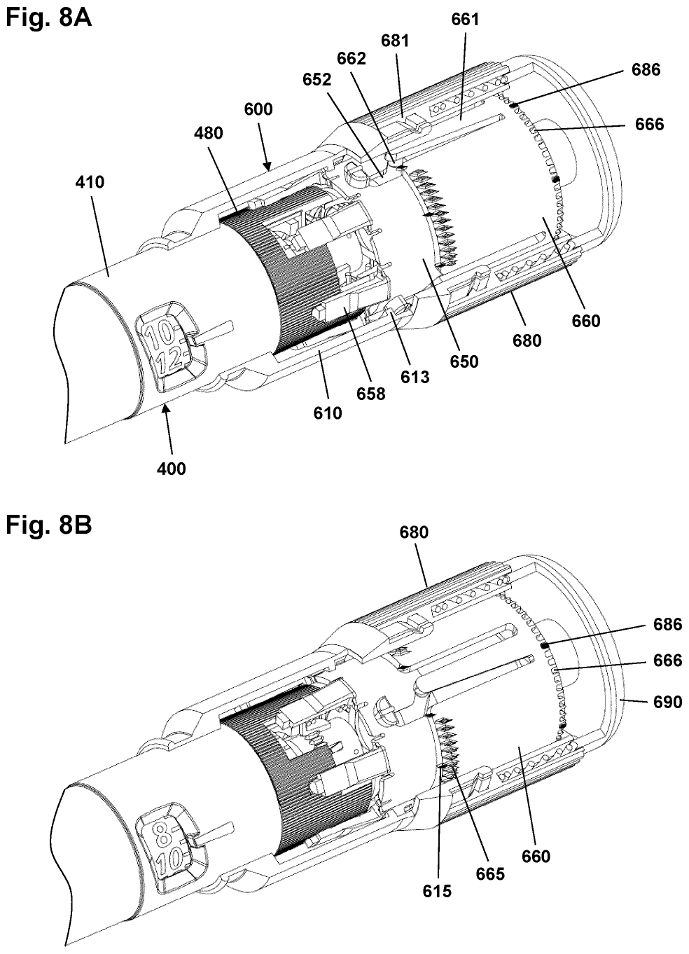

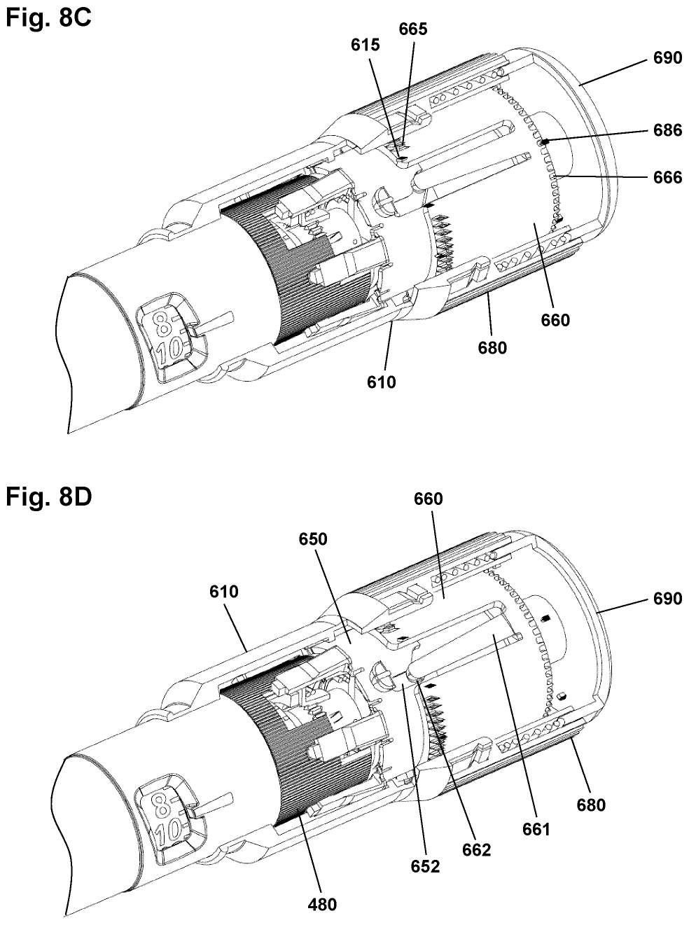

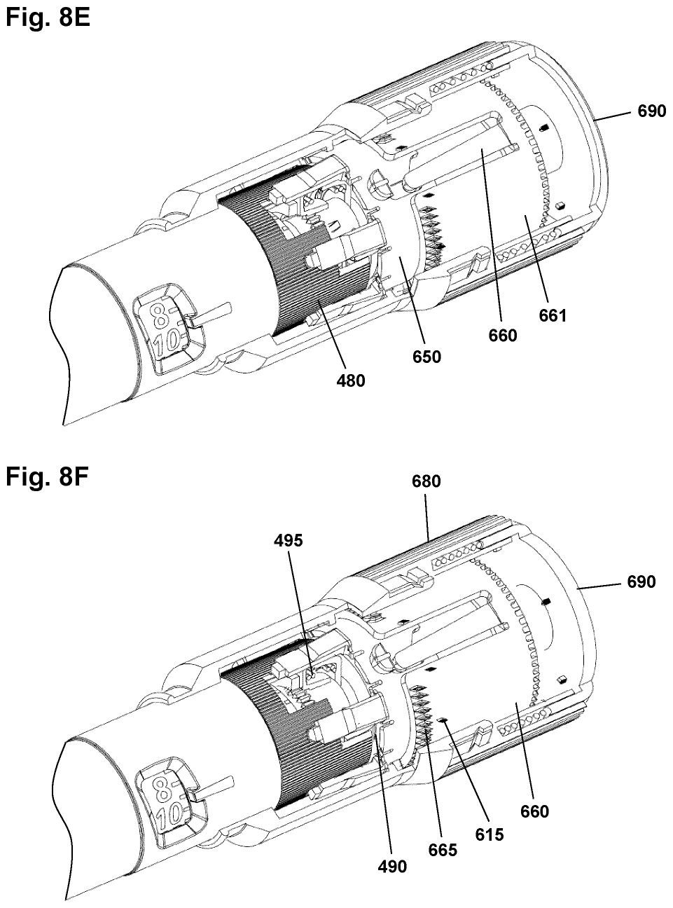

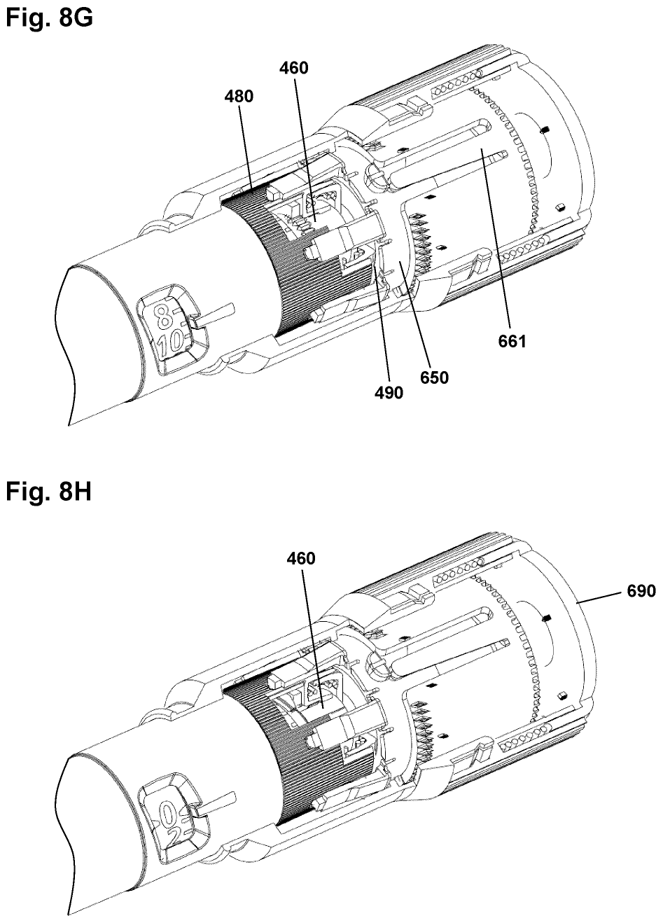

In FIG. 7A the housing member 610 having a general tubular configuration is shown. The distal end comprises coupling means (not shown) adapted to fixedly but releasably engage corresponding coupling means on the pen body. The proximal portion comprises a circumferential groove 611 and a number of axially oriented slots allowing the dial member 680 to be snap-mounted thereon. The housing member inner surface comprises a circumferential array of protrusions 615 adapted to engage with corresponding protrusions on the rotor member during operation, as well as a circumferential flange 613 (see FIG. 8A) for rotationally supporting the drive member 650 during dose setting.

The generally cylindrical dial member 680 shown in FIG. 7B comprises a distal inner circumferential fridge 681 (see FIG. 8A) adapted to be secured in axial direction in the housing member groove 611 while being able to rotate in both directions around its center axis. Circumferentially arranged protrusions 686 on the inside of the dial member near the proximal end are adapted to engage with the rotor member 660 and transmit rotational movement from the dial member to the rotor member when the dose release member 690 is fully released. The dial member further comprises a circumferential cavity 682 in which the dose release push-button 690 can be secured and axially guided (by not shown coupling means) and in which a coil return spring 640 (see FIG. 6B) for the dose release member can be accommodated.

The generally cup-shaped rotor member 660 shown in FIG. 7C comprises a proximal disc-formed portion and a distally extending generally cylindrical skirt portion. The skirt portion comprises a number (here: 3) of distally extending flexible arms 661 each having at the distal end an inwards protruding generally cylindrical follower structure 662 adapted to engage corresponding slot/track structures 652 in the drive member 650, the arms acting to initially transfer rotational movement and subsequently to absorb excess rotational movement thereby providing a controlled amount of torque to absorb slack as set out in greater detail below. The rotor member 660 comprises a proximally arranged circumferential toothing pattern 666 adapted to engage and disengage with the dial member protrusions 686 during operation. At the distal end the rotor member comprises a circumferential array of protrusions 665 arranged to engage and disengage with the housing member protrusions 615 and thereby lock the rotor member rotationally but not axially during operation. The disc-formed portion comprises a central opening 667 serving to keep the ESA axially centred via the securing pin 675. Friction surfaces 668 surrounding the opening on both sides allows the dose release member and the ESA to act as brake pads and lock the rotor member and the ESA rotationally to each other when the dose release member 690 and the ESA are pressed against the friction surfaces from each side.

The drive member 650 shown in FIG. 7D comprises a proximal generally cylindrical portion 651 from which a number of axially oriented drive fingers 658 extend in the distal direction, the drive fingers being adapted to engage the pen dial grooves 488 (see FIG. 5A) to thereby rotationally lock the drive member to the pen dial member 480. The proximal portion comprises a number (here: 3) of proximally open non-straight slots/tracks 652 adapted to receive and axially guide the rotor flexible arm followers 662. The proximal portion further comprises a number of circumferentially arranged guide structures 653 adapted to slidingly engage the housing inner wall surface as well as the proximally oriented support surface of the circumferential flange 613, this ensuring centering within the housing during rotational movement of the drive member.

With reference to FIGS. 8A-8H operation of the add-on device 600 will be described.

In general, FIGS. 8A-8H all show in a partial cut-away presentation the add-on device 600 of FIG. 6A mounted on the pen-formed drug delivery device 400 of FIG. 5A with the add-on housing member 610 being mounted on the pen housing 410 and with the add-on driver 650 rotationally coupled to the pen dial member 480 via the distally extending arms 658.

More specifically, in order to disclose operation of the internal components during operation of the add-on device, the housing member 610, the add-on dial member 680, the add-on release member 690 as well as the pen device dial member 480 has been partially cut away, however, it should be noted that the housing member projections 615 and the dial member projections 686 are not cut away.

Turning to FIG. 8A the add-on device 600 and the pen device 400 are shown in a state in which a dose of 11 IU has been dialled by a user, whereby all rotational slack in the pen dial mechanism has been absorbed in clockwise direction and all slack is left in counter-clockwise direction, the pen dial member 480 being the first pen component in the line of involved pen components. In the shown dose setting state the add-on dial member 680 is rotationally coupled to the rotor member 660 via the dial member projections 686 engaging the toothing pattern 666, and the rotor member 660 is rotationally coupled to the drive member 650 via the flexible arm follower structures 662 engaging the proximal portions of the track structures 652 in the drive member 650, this allowing the rotational movement of the add-on dial member 680 to be transferred to pen dial member 480 to set a dose. As appears, the “follower” is the active member and the track member is the passive member being moved. It is to be noticed that the rotational force will be transmitted via the flexible arms 661, however the introduced flexibility will hardly be noticed by the user when setting a dose.

In a pen device being dialed up to 11 IU all slack in the pen dial mechanism will be left in counterclockwise direction. If this is the required dose and injection is performed at this point, all slack is already eliminated or absorbed during the dial-up and the pen device dial member 480 and the drive member 650 will not be able to turn further clockwise. Thus, the flexible elements of the rotor member will only induce torque but no turning of the pen input dial 480, the turning being absorbed by the flexible arms serving as a torque limiter. The tension in the flexible arms 661 will be released at dose-release when the pen device dial is disengaged from the pen reset tube 160 and the pen device dial 480 and drive member 650 will turn clockwise without affecting any other parts until all tension in the flexible arms is released.

If instead the dialed dose of 11 IU has to be dialed down to 9 IU (see FIG. 8B) slack is now picked up in counterclockwise direction and all slack in the dial mechanism is now left in clockwise direction. During both dial-up and dial-down the rotor member 660 is rotationally locked to the add-on dial member 680.

When the corrected dose of 9 IU has been dialed, the user starts to move the add-on dose release button 690 in the distal direction. As the rotor member 660 moves axially with the dose release button the toothing 666 starts to slide out of engagement with the protrusions 686 in the dial member and the protrusions 665 on the distal part of the rotor starts sliding into engagement with the protrusions 615 on the inside of the housing member, see FIG. 8C. Thus, the rotor member 660 is briefly rotationally connected to both the housing member 610 and the dial member 680 and the rotor member is rotationally locked in the position in which it was left at dose setting. Slack remains being left in clockwise direction.

As the release button 690 is moved further distally, the rotor member 660 is disengaged from the dial member 680 but is still rotationally locked to the housing member 610. Due to the rotor follower structures 662 being moved distally into the curved portions of the track structures 652 in the drive member 650 a torque is now induced and the drive member starts to turn the pen dial member 480 and pick-up slack in clockwise direction.

As the release button 690 is moved yet further distally, the rotor member 660 and the ESA continue to move forward and the induced torque continues to turn the drive member 650 and pen dose dial 480 until all slack in the dose setting mechanism in the pen device is picked up. When all slack has been picked-up in clockwise direction and left in counter-clockwise direction further rotation of the pen device dial 480 requires the pen ratchet mechanism to move and the adjusted dose to be increased a unit. This however requires a significantly larger torque than the flexible arms 661 of the rotor member can provide. Correspondingly, rotation of the drive member 650 and the pen device dial member 480 stop which results in the flexible arms 661 starting to bend and thus take up further rotational movement induced by the rotor member 660, see FIGS. 8D and 8E in which the rotor flexible arms 661 have been bend thereby serving as a torque limiter.

As the ESA engages the pen device dose release button 490 the rotor member 660 disengages the housing member, i.e. the rotor protrusions 665 disengage the housing protrusions 615. Rotation of the ESA and the rotor member 660 is now prevented by the friction between the add-on dose release member 690, the rotor member 660 and the ESA, see FIG. 8F.

Alternatively, the protrusions 665 on the lower part of the rotor member may be made longer such that the rotor member 660 does not disengage the housing protrusions 615 and is not allowed to rotate before the add-on dose release member 690 is released and the rotor member has returned to its start-position in which it is engaged with the add-on dial member 680.

As the ESA engages the pen device dose release button 480 and starts to compress the pen device dose release return spring 495, the actuation switch on the ESA is activated and the ESA starts to perform measurements. The initial position of the pen device reset tube is then established prior to out-dosing.

When the pen device dose release button 490 has been moved sufficiently in the distal direction by the ESA, the pen reset tube 460 disengages from the pen device dial member 480 which can then rotate freely. This allows the build-up tension in the flexible arms 661 of the rotor member to be released by rotating the drive member 650 and the disengaged pen device dial member 480 in clockwise direction, see FIG. 8G.

When the reset tube 460 has disengaged the coupling with the pen device housing, the reset tube starts to turn counter-clockwise as the out-dosing starts. The ESA continues to perform multiple measurements of reset tube angular position during out-dosing in order to establish the number of complete revolutions of the reset tube during out-dosing. When out-dosing is completed (see FIG. 8H) the reset tube stops at its zero-position (or at a position at which the add on dose release button 690 was released and out-dosing stopped) and the last measured rotational position of the reset tube when the ESA switch is deactivated is obtained and used as end position whereby the out-dosed volume can be calculated as described above.

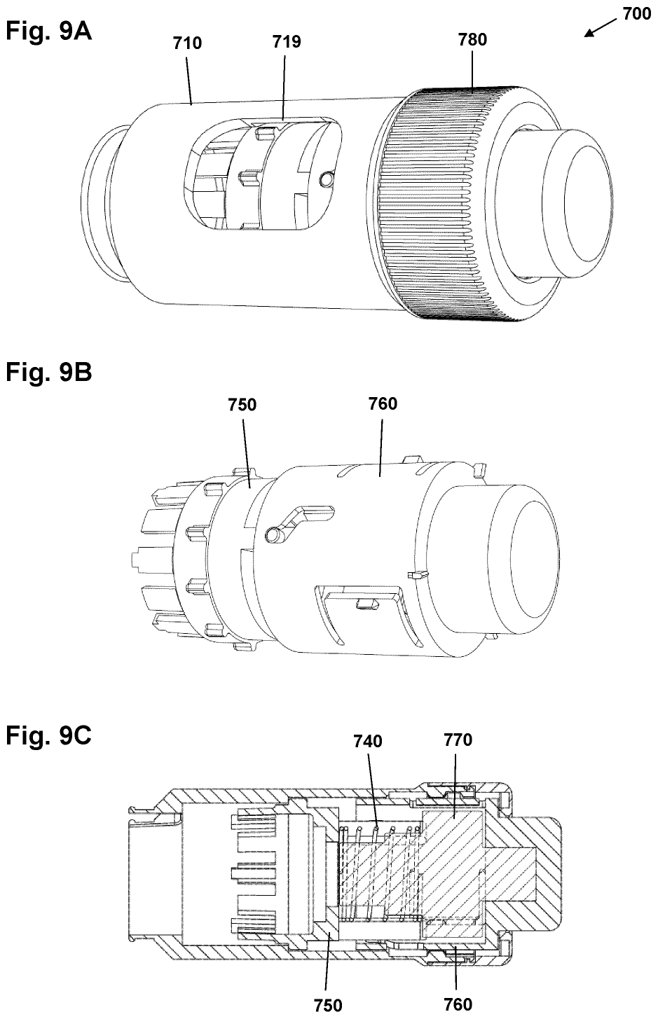

Turning to FIGS. 9A-9C a second exemplary embodiment of a slack counter-acting mechanism suitable for incorporation into an add-on dose logging device of the general type disclosed in FIG. 5B will be described.

FIG. 9A shows an add-on dose logging device 700 comprising an outer assembly and a thereto coupled inner assembly. The outer assembly comprises a housing member 710 adapted to be coupled to the pen housing and a thereto rotationally coupled add-on dial member 780. The housing is shown with a window 719 for illustrative purposes only. FIG. 9B shows the inner assembly comprising a drive member 750 adapted to non-rotationally engage the pen device dial member and a combined a rotor/button member 760 arranged to transfer rotational movement from the dial member 780 to the drive member 750 during dose setting and to provide slack counter-acting rotation during dose release, as well as an electronic sensor assembly (ESA) 770 (see FIG. 9C) arranged in the hollow interior of the rotor/button member 760. In FIG. 9C the ESA 770 is shown arranged inside the rotor/button member, a biasing spring 740 being arranged between the ESA and the drive member 750 to provide a return force to the rotor/button member when the latter is moved distally by a user.

Before describing the working principle of the add-on device 700 the individual cooperating members will be described in greater detail.

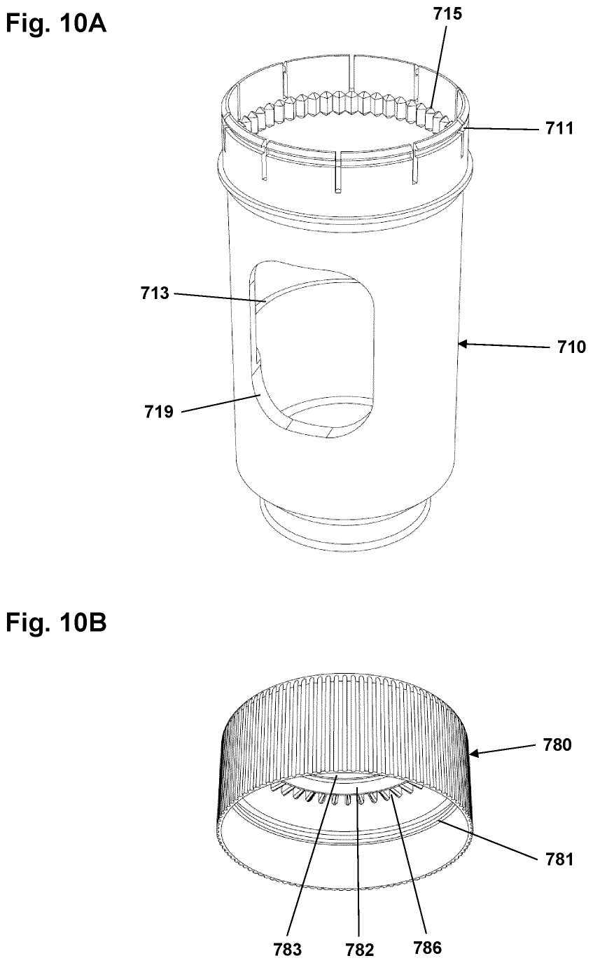

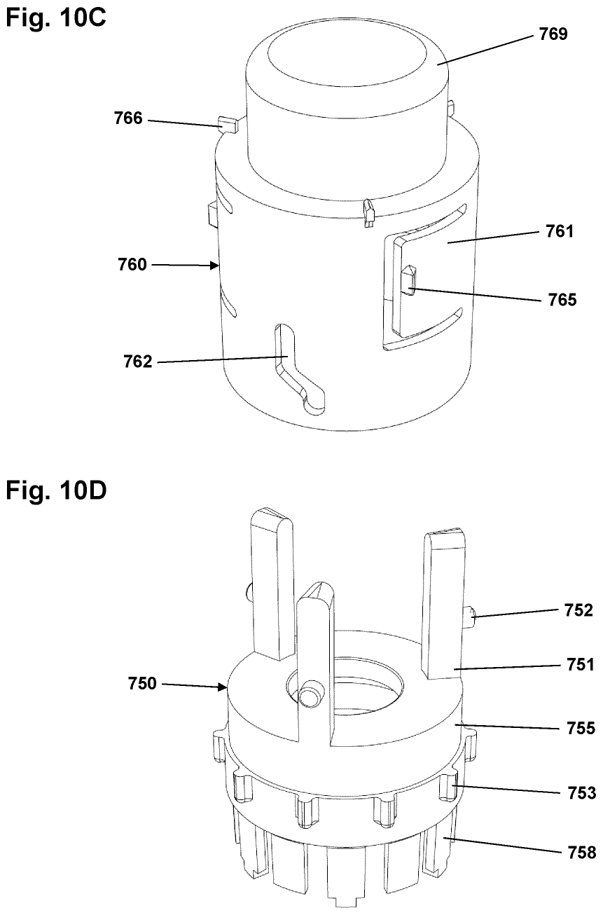

In FIG. 10A the housing member 710 having a general tubular configuration is shown. The distal end comprises coupling means (not shown) adapted to fixedly but releasably engage corresponding coupling means on the pen body. The proximal portion comprises a circumferential groove 711 and a number of axially oriented slots allowing the dial member 780 to be snap-mounted thereon. The housing member inner surface comprises a proximally arranged circumferential array of protrusions 715 adapted to engage with corresponding protrusions on the rotor/button member 760 during operation, as well as a circumferential proximally facing edge 713 for rotationally supporting the drive member 750 during dose setting. The housing is shown with a window 719 for illustrative purposes only.

The generally cylindrical dial member 780 shown in FIG. 10B comprises an inner circumferential ridge 781 adapted to be secured in axial direction in the housing member groove 711 while being able to rotate in both directions around its center axis. The proximal portion comprises a circumferential wall portion 782 surrounding a circular opening 783 adapted to receive the proximal portion of the rotor/button member 760. On the distal surface of the circumferential wall portion a circumferential toothing 786 is provided adapted to engage corresponding projections on the rotor/button member 760 and transmit rotational movement from the dial member 780 to the rotor/button member during dose setting.

The generally cylindrical rotor/button member 760 shown in FIG. 100 comprises a proximal button portion 769 with a diameter corresponding to the dial member opening 783 and a distally extending generally cylindrical rotor/skirt portion having a larger diameter. The skirt portion comprises a number (here: 3) of circumferentially extending flexible fingers 761 each having at the free end an outwards oriented protrusion 765 adapted to engage the circumferential housing toothing 715 to thereby provide a torque limiter as set out in greater detail below. The rotor/button member 760 comprises a number (here: 3) of circumferentially arranged proximally extending projections 766 adapted to engage and disengage with the dial member toothing 786 during operation. At the distal end the rotor portion comprises a number (here: 3) of curved/non-straight slots/tracks 762 adapted to receive and axially guide the drive member followers 752.

The drive member 750 shown in FIG. 10D comprises a generally cylindrical main portion 755 from which a number of axially oriented drive fingers 758 extend in the distal direction, the drive fingers being adapted to engage the pen dial grooves 488 to thereby rotationally lock the drive member to the pen dial member 480. A number of arms 751 (here: 3) extends axially in the proximal direction, each arm comprising an outwards protruding generally cylindrical follower structure 752 adapted to engage corresponding slot/track structures 762 in the drive member 760. The main portion further comprises a number of circumferentially arranged guide structures 753 adapted to slidingly engage the housing inner wall surface as well as the proximally oriented support surface of the circumferential edge 713, this ensuring centering within the housing during rotational movement of the drive member. The ESA (see FIG. 9C) comprises axially oriented slots adapted to receive the arms to their rotationally couple the ESA to the drive member.

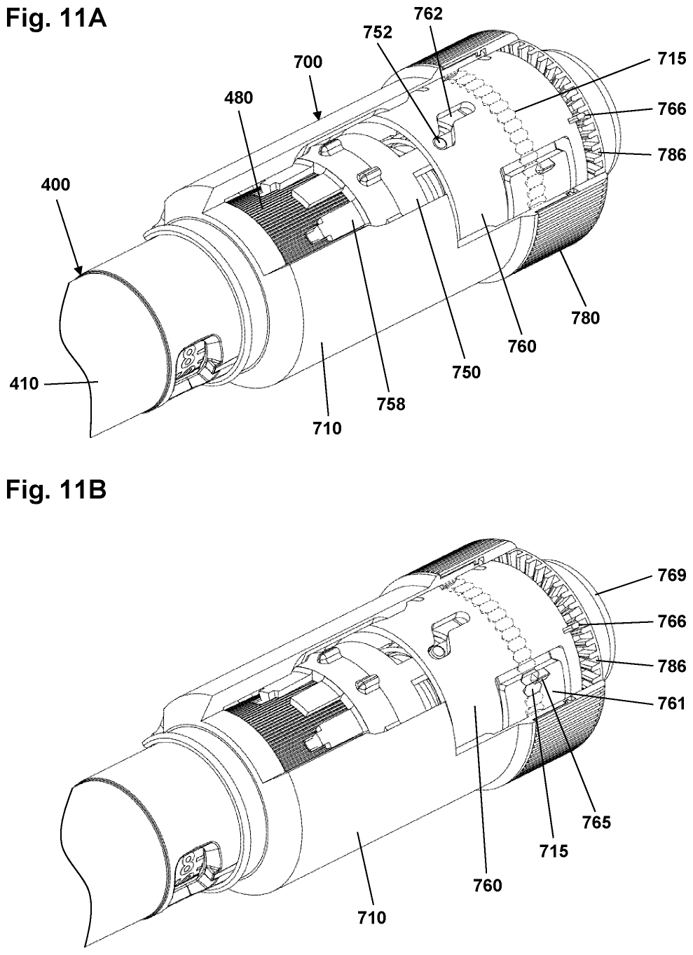

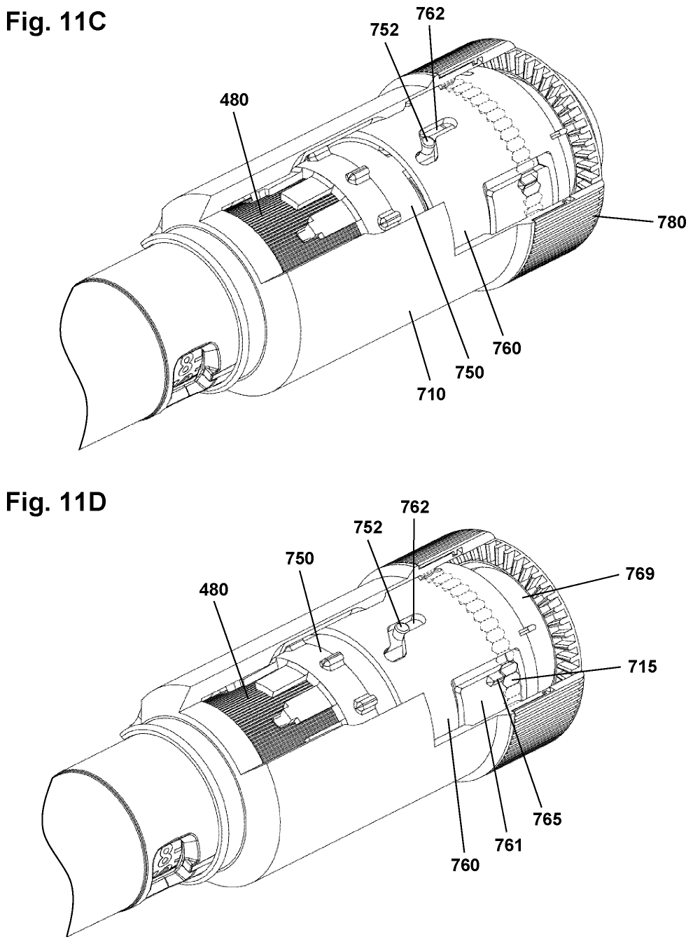

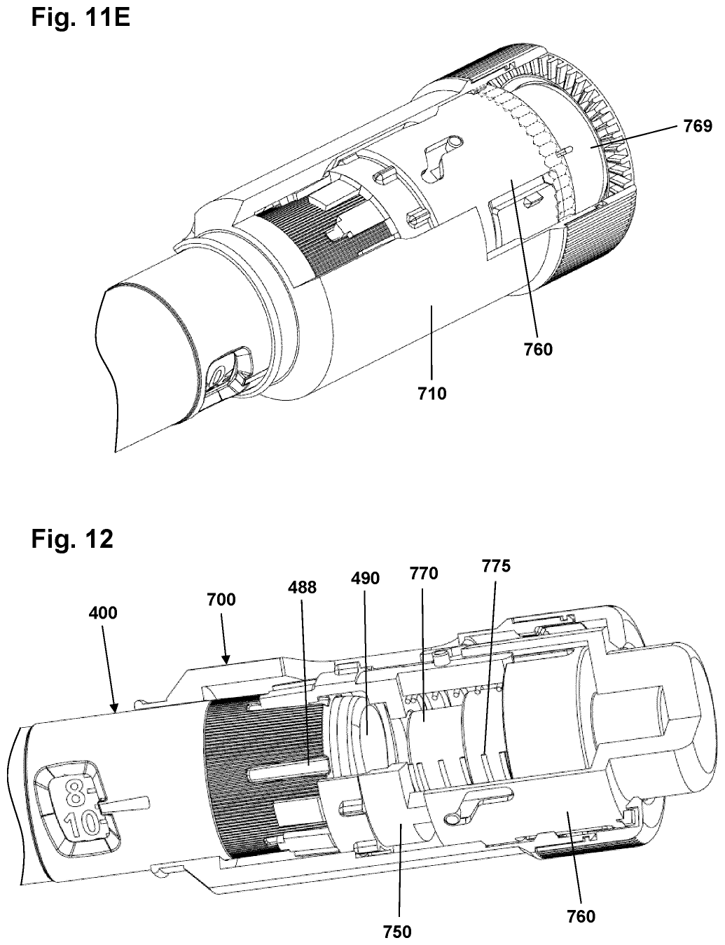

With reference to FIGS. 11A-11E operation of the add-on device 700 will be described.

In general, FIGS. 11A-11E all show in a partial cut-away presentation the add-on device 700 of FIG. 9A mounted on the pen-formed drug delivery device 400 of FIG. 5A with the add-on housing member 710 being mounted on the pen housing 410 and with the add-on driver 750 rotationally coupled to the pen dial member 480 via the distally extending fingers 758.

More specifically, in order to disclose operation of the internal components during operation of the add-on device, the housing member 710 and the add-on dial member 780 have been partially cut away, however, it should be noted that the housing member projections 715 are not cut away.

The fundamental working principle for the second embodiment is the same as for the first embodiment, where a torque in counter-clockwise direction (dial-up direction) is always applied on the pen device input dial member when the add-on dose release button is actuated. In this way any slack will always be left to the same side, regardless of size of actual slack in the particular device or whether the user dialed up or down just prior to out-dosing. If the actual slack is less than tried compensated for, or the user dialed up just prior to injection, the torque limiter between the add-on housing and rotor/button member will be activated and slip. Thus, rather than dial the device up a unit the rotor/button member will skip one or more clicks in the rotational torque limiter and rotate counter-clockwise instead of turning the drive member further clockwise.

The add-on dose release button is integrated in the rotor in the second embodiment. As the dose release button is pushed, the tracks in the rotor/button member moves down whereby a torque is induced in the drive member, causing it to turn the input dial of the injection device in clockwise (dial-up) direction and pick-up any slack.

The protrusions on the top edge of the rotor/button member and the toothing in the dial member are to allow the rotor/button member and thus the drive member to be operated by the add-on input dial member 780 during dose size setting. The protrusions 765 on the flexible fingers 761 on the rotor/button member and the protrusions 715 in the housing member 710 are designed to lock the rotor/button rotationally to the add-on housing during axial activation of the dose release button, i.e. until a given amount of torque is generated in which case the “lock” will jump to the next protrusion.

Turning to FIG. 11A the add-on device 700 and the pen device 400 are shown in a state in which an initially dialled dose of e.g. 11 IU has been dialed down to 9 IU. Thus, initially all rotational slack in the pen dial mechanism has been absorbed in clockwise direction and all slack is left in counterclockwise direction. However, when dialing down to 9 IU all slack in the dial mechanism is left in clockwise direction. During both dial-up and dial-down the add-on dial member 780 is rotationally coupled to the rotor/button member 760 via the dial member toothing pattern 786 engaging projections 766, and the rotor/button member 760 is rotationally coupled to the drive member 750 via the arm follower structures 752 engaging the distal portions of the track structures 762 in the rotor/button member 760, this allowing the rotational movement of the add-on dial member 780 to be transferred to pen dial member 480 to set or adjust a dose.

When the corrected dose of 9 IU has been dialed, the user starts to move the rotor/button member 760 in the distal direction by pushing down on the release button portion 769, see FIG. 11B. As the rotor/button member starts to move distally the rotor projections 766 start to disengage the dial member toothing 786 and the protrusions 765 on the flexible fingers 761 start to slide into engagement with the circumferential protrusions 715 on the inside of the housing member 710. Thus, the rotor/button member 760 is briefly rotationally connected to both the housing member and the dial member and thus rotationally locked in the position in which it was left at dose setting.

As the rotor/button member 760 is moved further distally it disengages from the dial member 780 but is still rotationally locked to the housing member 710 as shown in FIG. 11C. Due to the drive member followers 752 arranged in the curved tracks 762 of the axially moving rotor/button member a torque is now induced in the drive member 750 which starts to turn the pen device dial member 480 and pick-up slack in clockwise direction

During continued actuation of the release button portion 769 the rotor/button member 760 and the thereto coupled ESA continues to move distally as shown in FIG. 11D, this resulting in an increasing torque being applied to the drive member as the drive member followers 752 is moved laterally in the non-axial portion of the rotor track 762, the drive member continuing to turn the pen device dial member 480 and pick-up slack in clockwise direction. However, when the torque transferring limit between the finger protrusions 765 and the housing protrusions 715 is reached the rotational lock disengages as the flexible fingers 761 flex inwards resulting in the rotor protrusion “jumping” one increment on the housing toothing 715. This limit should be designed such that even a worst-case magnitude of slack is picked up within a safe margin.

If less than worst-case slack is present in the actual device or if the device was dialed up, and not down, just prior to activation of the rotor/button member 760, all slack will be picked-up in clockwise direction and left in counter-clockwise direction prior to disengagement of the rotational lock. In this case, further rotation of the pen device dial member 480 requires the ratchet mechanism to move and the adjusted dose to be increased a unit.

This however requires a significantly larger torque than the rotational lock of the flexible fingers 761 of the rotor/button member can support and instead the protrusions 765 on the flexible elements of the rotor/button member will flex inwardly. This will allow the rotor/button member 760 to rotate one or more clicks counterclockwise to compensate for the clockwise rotation of the drive member 750 relative to the rotor/button member. This may cause the drive member to be dialed a little counter-clockwise and re-introduce a little slack in clockwise direction, since the rotor/button member will rotate counter-clockwise in an integer number of clicks.

However, the rotor/button member 760 will only skip clicks as long as the track of the rotor/button member induces a torque in the drive member 750, while the protrusions of the rotor/button member is engaged with the toothing/protrusions in the housing. Thus, any small counter-clockwise rotation of the drive member caused by a click-back of the rotor/button member 760 will immediately be followed by a resumed clockwise rotation where the re-introduced slack is being picked up.

As the ESA engages the pen dose release button 490 and starts to compress the pen device dose release button return spring, the actuation switch on the ESA is activated and the ESA starts to perform measurements to determine the initial rotation position of the pen reset tube prior to out-dosing. When the pen device dose release button 490 has been moved sufficiently in the distal direction by the ESA, the pen reset tube 460 disengages from the pen device dial member 480 which can then rotate freely. This allows any build-up tension in the torque limiter to be released by rotating the drive member 750 and the disengaged pen device dial member 480 in clockwise direction.

When the pen device reset tube has disengaged the coupling with the pen device housing, the reset tube starts to turn counter-clockwise as the out-dosing starts. The ESA continues to perform multiple measurements of reset tube angular position during out-dosing in order to establish the number of complete revolutions of the reset tube during out-dosing. When out-dosing is completed (see FIG. 11E) the reset tube stops at its zero-position (or at a position at which the add on dose release button 769 was released and out-dosing stopped) and the last measured rotational position of the reset tube when the ESA switch is deactivated is obtained and used as end position whereby the out-dosed volume can be calculated as described above.

When the user releases pressure on the dose release button portion 769 the rotor/button member 760 and the ESA returns to their initial proximal position by means of the return spring 740 shown in FIG. 9C.

In FIG. 12 the add-on device 700 and the pen device 400 of FIG. 11A are shown in a different view in which the rotor/button member 760 and the drive member 750 have been partly cut away, this allowing the ESA 770, the return spring 740, the pen dial grooves 488 and the pen device release member 490 to be clearly shown.

In the above description of exemplary embodiments, the different structures and means providing the described functionality for the different components have been described to a degree to which the concept of the present invention will be apparent to the skilled reader. The detailed construction and specification for the different components are considered the object of a normal design procedure performed by the skilled person along the lines set out in the present specification.

1. An add-on device adapted to be releasably mounted on a drug delivery device, the drug delivery device comprising:
a housing defining a reference axis,
a drug reservoir or structure for receiving a drug reservoir, and
drug expelling structure comprising:
a dose setting member adapted to rotate in a first direction to incrementally set a dose, and rotate in an opposed second direction to incrementally reduce a set dose, the dose setting member being arranged at the proximal end of the housing, and
a release member actuatable between a proximal position and a distal position, the proximal position allowing a dose amount to be set, the distal position allowing the drug expelling structure to expel a set dose,
the add-on device comprising:
an add-on housing adapted to be releasably attached to the drug delivery device housing in an axially and rotationally non-moveable position,
a drive member adapted to be mounted rotationally locked on the dose setting member,
an add-on dose setting member coupled to the add-on housing rotatable free but axially locked and adapted to rotate in the first direction to set a dose, and rotate in the opposed second direction to reduce a set dose,
an actuatable add-on release member axially moveable relative to the add-on dose setting member between:
(i) a proximal dose setting position in which the add-on dose setting member can be operated to rotate the drive member and thereby, when mounted on the dose setting member, set a dose, and
(ii) a distal dose expelling position in which the release member, when the add-on device is mounted on the drug delivery device, is moved to its distal position to release a set dose,
a linear-to-rotational converter mechanism adapted to convert axial movement of the add-on release member, when the add-on release member is moved from the proximal position towards the distal position, to rotational movement of the drive member in the first direction,
whereby, when the add-on device is mounted on the drug delivery device:
the dose setting member is biased in the first direction by the drive member when the add-on release member is moved from the proximal position towards the distal position.

2. The add-on device as in claim 1, further comprising:
a torque limiter preventing, when the add-on device is mounted on the drug delivery device, that a torque above a pre-set level can be transferred to the drive member when the add-on release member is moved from the proximal position towards the distal position.

3. The add-on device as in claim 2, wherein the torque limiter comprises:
a flexible element adapted to deform when a torque above the pre-set level has been transferred to the drive member, or
is in the form of a ratchet torque limiter.

4. The add-on device as in claim 1, wherein the rotational movement is generated by a cam-follower mechanism.

5. The add-on device as in claim 4, wherein the cam-follower mechanism further comprises a track and a follower arranged in the track, the track having a radial component, the follower and track moving axially relative to each other member when the add-on release member is moved from the proximal position towards the distal position.

6. The add-on device as in claim 5, wherein the follower comprises a flexible arm adapted to bend when a torque above a pre-set level has been transferred to the drive member.

7. The add-on device as in claim 1, the drug expelling structure comprising:
an indicator adapted to rotate during expelling of a dose amount, the amount of rotation being indicative of the size of the expelled dose amount, and
the add-on device comprising:
sensor structure operatable to detect the amount of rotation of the indicator during expelling of a dose amount,
wherein:
rotational movement has been applied to the drive member prior to the sensor structure being operated,
whereby:
when the add-on device is mounted on the drug delivery device, it is assured that rotational slack in the drug expelling structure has been removed by rotation of the dose setting member in the first direction.

8. The add-on device as in claim 7, further comprising an actuator structure adapted to move axially and engage the release member when the add-on release member is actuated, wherein the sensor structure is coupled to and moves axially with the actuator structure.

9. The assembly comprising a drug delivery device as defined in claim 1 and an add-on device as defined in claim 1.

10. The assembly comprising a drug delivery device and add-on device as defined in claim 7.

11. The assembly as in claim 10, wherein:
the indicator comprises a plurality of dipole magnets, and
the sensor comprises:
a plurality of magnetometers arranged non-rotational relative to the housing in a mounted state and adapted to determine magnetic field values from the plurality of dipole magnets, and
processor structure configured to determine on the basis of measured values from the plurality of magnetometers a rotational position and/or a rotational movement of the indicator.

12. The assembly comprising a drug delivery device and add-on device as defined in claim 1, wherein:
the drug expelling structure comprises an indicator adapted to rotate during expelling of a dose amount, the amount of rotation being indicative of the size of the expelled dose amount, and
the drug delivery device is adapted for mounting of a dose logging add-on device comprising sensor structure operatable to detect the amount of rotation of the indicator during expelling of a dose amount.

13. The assembly comprising a drug delivery device and add-on device as defined in claim 1, wherein:
the drug expelling structure comprises an indicator adapted to rotate during expelling of a dose amount, the amount of rotation being indicative of the size of the expelled dose amount, and
the assembly further comprises a dose logging add-on device adapted to be mounted on the drug delivery device, comprising sensor structure operatable to detect the amount of rotation of the indicator during expelling of a dose amount.

14. The assembly as in claim 13, wherein:
the indicator comprises a plurality of dipole magnets, and
the sensor structure comprises:
a plurality of magnetometers arranged non-rotational relative to the housing in a mounted state and adapted to determine magnetic field values from the plurality of dipole magnets, and
processor structure configured to determine on the basis of measured values from the plurality of magnetometers a rotational position and/or a rotational movement of the indicator.

15. A unitary drug delivery device, comprising:
a housing defining a reference axis,
a drug reservoir or structure for receiving a drug reservoir,
drug expelling structure comprising:
an inner dose setting member adapted to rotate in a first direction to set a dose, and rotate in an opposed second direction to reduce a set dose,
an inner release member actuatable between a proximal position and a distal position, the proximal position allowing a dose amount to be set, the distal position allowing the drug expelling structure to expel a set dose, and
an indicator adapted to rotate during expelling of a dose amount, the amount of rotation being indicative of the size of the expelled dose amount,
a user dose setting member coupled to the housing rotatable free but axially locked and adapted to rotate in the first direction to set a dose, and rotate in the opposed second direction to reduce a set dose,
an actuatable user release member axially moveable relative to the user dose setting member between (i) a proximal dose setting position in which the user dose setting member can be operated to rotate the inner dose setting member to set a dose, and (ii) a distal dose expelling position in which the inner release member is moved to its distal position to release a set dose, and
a linear-to-rotational converter mechanism adapted to convert axial movement of the user release member, when the user release member is moved from the proximal position towards the distal position, to rotational movement of the inner dose setting member in the first direction.
